(19) United States
(12) Patent Application Publication
(10) Pub. No.: US 2025/0345084 A1
(43) Pub. Date: Nov. 13, 2025

(54) ENDOSCOPIC TOOLS WITH SOFT TIP AND METHOD

(71) Applicant: ENDOSCOPYTOOLS, LLC, Carmel, IN (US)

(72) Inventor: Mazen Alsatie, Carmel, IN (US)

(21) Appl. No.: 19/279,370

(22) Filed: Jul. 24, 2025

Related U.S. Application Data
(63) Continuation of application No. 17/251,894, filed on Dec. 14, 2020, now abandoned.
(60) Provisional application No. 62/684,396, filed on Jun. 13, 2018.
(60) Provisional application No. 62/743,226, filed on Oct. 9, 2018.

(51) Int. Cl. A61B 17/29; A61B 1/00; A61B 1/018; A61B 17/00; A61B 17/3201

(52) U.S. Cl. CPC A61B 17/29; A61B 1/00087; A61B 1/018; A61B 17/3201; A61B 2017/00862; A61B 2017/2905; A61B 2017/2939

(57) ABSTRACT

An endoscopic tool comprises a manipulator coupled to a distal instrument via a filament or filaments such that the instrument is directed transversely to the longitudinal axis of the endoscope. For example, a snare, wire or cannula extends transversely from a tubular lumen of the tool. The distal instrument may comprise grasping jaws, and a surface of the grasping jaws may be of an elastomeric polymer.

### CROSS REFERENCE TO RELATED APPLICATIONS

This application is related to two U.S. provisional applications: provisional No. 62/743,226 filed Oct. 9, 2018 and provisional No. 62/684,396 filed Jun. 13, 2018, the disclosures of both herein being incorporated by reference in their entirety.

### FIELD

The field relates to endoscopic procedures and tools.

### BACKGROUND

An endoscope is an known instrument inserted into a body cavity to view the cavity and to carry out certain surgical, or other medical, procedures. Food impaction in the esophagus is a common gastrointestinal emergency in which food gets stuck and must be dislodged using a surgical procedure. There are several tools for removal of impacted food. The procedure for food removal can be tedious and several tools may be utilized. Cutting the food bolus into smaller pieces has proven to be a generally safe method to relieve the impaction. With current tools, this process may be laborious. Regular snares are not often unsuccessful. Worse still, the instruments may cause damage to surrounding tissues. Another use of an endoscopic snare is removal of unwanted tissue within the gastrointestinal tract such as polyps or other growths. Known tools have difficulty accessing and removing polyps or residual tissue that is not readily accessible.

It is known to use controls at the proximal end of the instrument may control the instrument's tip through cables or filaments running through the endoscope's tubular structure. These controls provide some modest control over the disposition of the distal tip of an instrument, often resulting in a laborious trial and error effort to position the instruments correctly within a cavity in order to perform a desired surgical procedure.

Endoscopic Retrograde Cholangiopancreatography (ERCP) is a procedure that combines the use of an endoscope and radiographically guided instruments to reach the ampulla and selectively cannulate the common bile duct, pancreatic duct, or both. Frequently, the patient's indications require only cannulation of the common bile duct without cannulation of the pancreatic duct. Achieving selective cannulation into the bile duct generally provides a higher overall success rate and shortened procedure time as well as lower unintended complications. Chief among these complications is pancreatitis. The major difficulty encountered involves the ability to direct the cannula in the necessary upward direction from the tip of the endoscope to engage the common bile duct and not enter or damage the pancreatic duct.

Current tools used for selective cannulation of the common bile duct utilize methods of wire insertion and catheter insertion as well as contrast injection and depend primarily on the skills of the person operating the endoscope. Endoscopic Retrograde Cholangiopancreatography (ERCP) is typically carried out with a side-view endoscope, such as illustrated in FIGS. 1 and 2.

Existing tools don't have a distal end that can be readily guided to the papilla so that the instrument can engage the opening and assist in teasing it partially open. The problem is that known instruments cannot obtain the proper angle to engage the common bile duct directly without unintentionally entering the pancreatic duct. This can lead to unwanted complications and unsatisfactory outcomes.

### SUMMARY

An endoscopic tool comprises a proximal end having a manipulator for manipulating an instrument disposed at a distal end. The tool having a filament such as a cable or wire connecting the proximal end to the distal end such that manipulating the manipulator operates the instrument at the distal end. Herein, the term “filament” is used as a broad term to mean any type of wire, cable or other flexible shaft that may be inserted through a tubular lumen to push or pull the instrument using the manipulator. In one example, a more rigid filament may be used to push and pull. in an alternative example, a filament may be more flexible and may be capable of only pulling against the tool (but not capable of pushing the instrument). A spring-like resilience may act to pull the instrument back into place after being pulled by a highly flexible filament into an arcuate shape, for example, even though the flexible filament would not be able to push the tool back to its original shape.

The instrument at the distal end may be used for probing, cutting and/or removing an object or orifice in a body cavity. For example, the body cavity may be an intestinal tract or an esophagus or the like. For example, the tool may be inserted through an endoscope, the endoscope having a means, as is well known in the art, for viewing inside of the body cavity.

For example, the instrument may have a flexible end with a snare disposed within a tubular lumen of the tool. The tubular lumen may have a soft tip extending distally from the tubular lumen, and the snare may comprise a loop that extends transversely to the shaft of the tubular lumen when the loop extends from an orifice in a side of the tubular lumen or a side of the soft tip extending from the tubular lumen. Herein “transverse” and “transversely” mean perpendicularly or obliquely extending from the longitudinal axis of the tubular lumen (as directed by the manipulator and the orifice, when extended from the orifice). “Soft” means a degree of softness capable of reducing damage to tissues when a soft component is pressed against mucous membranes and internal body cavities, as this would be understood by one of ordinary skill in the art. For example, a silicone rubber coating is soft, as are other elastomeric materials.

The loop, prior to extension from the orifice, is directed along the longitudinal axis, but as the loop is directed by the orifice, the loop may extend transversely from the side of the tubular lumen. The loop may be constructed from a flexible wire or a shape memory alloy. The loop may be coupled to a distal end of a cable, and a proximal end of the cable may be coupled to the manipulator of the tool.

For example, a distal tip of the tool may be comprised of soft blunt tip. A side opening in the tubular member or the soft blunt tip may be disposed proximate to the distal soft blunt tip. The soft blunt tip may be used to probe an orifice in order for the tool to enter the orifice, for example. The orifice in the body cavity may be a duct, such as a bile duct, for example.

For example, the orifice may be from 2 to 3 mm from a distal end of the soft blunt tip. In one example, the loop may extend from the orifice perpendicularly to the longitudinal axis of the tubular lumen. In some procedures, the transversely directed loop greatly improves the ability of a surgeon to quickly and accurately grasp, under direct visualization, an impacted bolus or polyp or the like, for example, while the soft blunt tip prevents inadvertent damage to mucus membranes, ducts, and other tissues within the body cavity. A loop may be used to dissect the bolus for its removal or to remove an impaction lodged in the body cavity, for example.

In another example, the tool may be used for cannulating a duct, such as a common bile duct, for example. The instrument at the distal end of the tool may be flexible and may allow for accurate positioning within the ampulla of Vater proximate the sphincter of oddi. A distal end of the instrument may probe and facilitate entry of a wire or cannula into an orifice. In one example, a secondary filament redirects the tip greater than 90 degrees from the longitudinal axis of the endoscope allowing the instrument to gently traction on the ampulla's upper rim, stabilizing and straightening the lumen for insertion of the wire or cannula.

For example, the instrument comprises grasping jaws coupled with the manipulator such that the grasping jaws engage a portion or papilla of the sphincter of oddi, teasing the papilla at least partially open for insertion of a wire or cannula. The wire or cannula may extend through an orifice extending longitudinally or transversely to the longitudinal axis of the tubular lumen of the tool, such that the wire or cannula is directed into the duct.

For example, the grasping jaws of the instrument may be made of a soft and slip resistant material, such as an elastomer or an elastomerically coated rigid material, allowing a firm grasp without cutting or otherwise tearing the tissue being grasped. For example, a non-slip silicone polymer may be used for coating a metal grasper. Grasping jaws may be coated with a silicone or other elastomeric polymer, for example. The grasping jaws may be made with one or both grasping jaws movable from an open position to a grasping position with a scissor mechanism or any other mechanism capable of closing the jaws in a grasping motion.

In one example, the tool further comprises a secondary filament coupled at one end near the tip of the tools distal end and coupled at the other end to the manipulator such that the secondary filament may be pulled by the manipulator such that the movement or bending of the distal tip arcuately disposes the distal end for accurately positioning the instrument within the body cavity. In this example, the secondary filament may be any material compatible with the instrumentation and its internal use and includes materials from the group consisting of a metal such as a wire or cable, fiber materials, and polymer materials. In one example, a titanium metal wire is selected.

By combining grasping jaws and a secondary filament the tip of the instrument extending from the endoscope is positioned independent of the endoscoe in an orientation more favorable for delivering a wire or a cannula from the tip of the instrument through the papilla teased open by the grasping jaws and into the common bile duct. For example, an orifice in a soft tip may be directionally angled for easy entry and extension of a wire or a cannula into the common bile duct, without inadvertently entering any other duct, such as the pancreatic duct. Alternatively, the wire or the cannula may be reoriented to direct the wire or cannula safely into the pancreatic duct, if desired, for diagnostic or corrective surgical procedures.

### BRIEF DESCRIPTION OF THE DRAWINGS

The following drawings are illustrative examples and do not further limit any claims that may eventually issue.

FIG. 1 illustrates a side-view endoscope of the prior art, providing common features not intended to limit the claims.

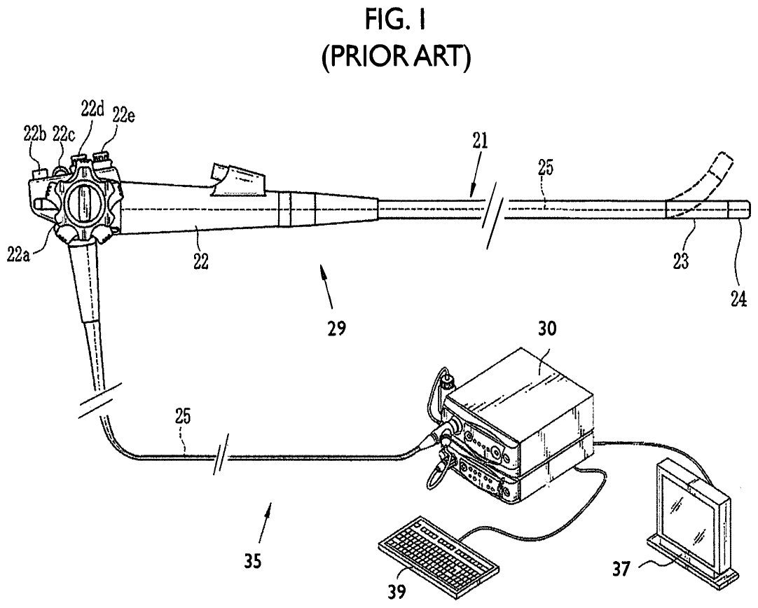

FIG. 2 illustrates a distal end or tip of a side-view endoscope such as illustrated in FIG. 1.

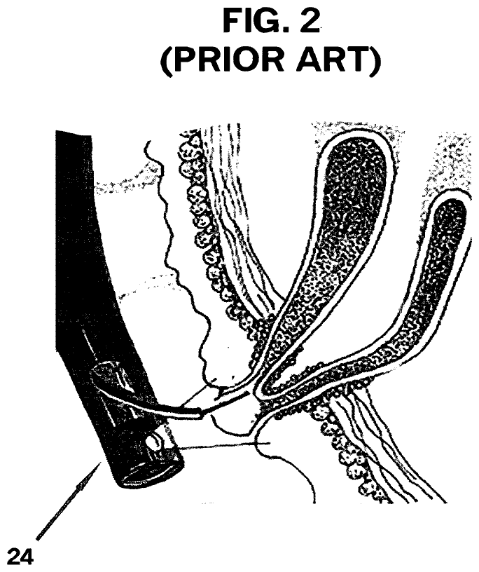

FIG. 3A-3D illustrates an example of a distal end or tip of a tool with (A) a bending portion of the tubular lumen with a grasping device 50 extending from the tip, (B) a detailed view of a mechanism for closing a pair of jaws of a grasping device, (C) a partial cross sectional view along an axis of the lumen showing an orifice on the side of tubular lumen/tip, and (D) an example showing a coupling member 57 and a mounting bracket 58 for the grasping jaws and an orifice extending through the tip without a bend for directing an instrument transversely.

FIG. 4 illustrates a sketch of the known human anatomy of the prior art.

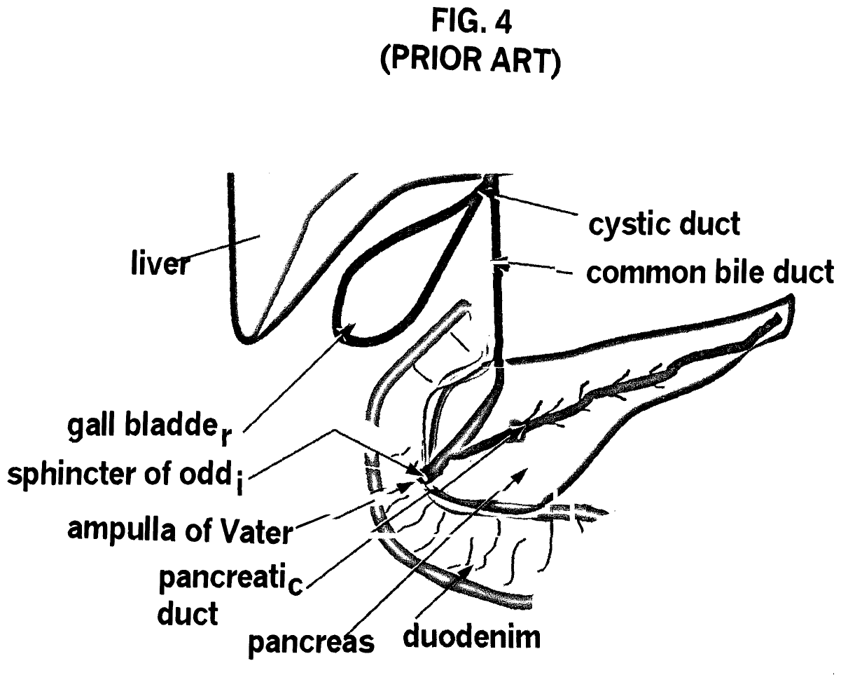

FIG. 5 illustrates an example of a distal end or tip of a tool with a snare loop extending transversely from the lumen/tip.

FIG. 6 illustrates a prior art sphincterotome.

When the same reference characters are used, these labels refer to similar parts in the examples illustrated in the drawings.

### DESCRIPTION

In one example, an endoscopic tool may comprise an instrument for use in existing endoscopes by inserting the instrument through a tubular channel of the endoscope and controlling the instrument using a manipulator as described in more detail elsewhere.

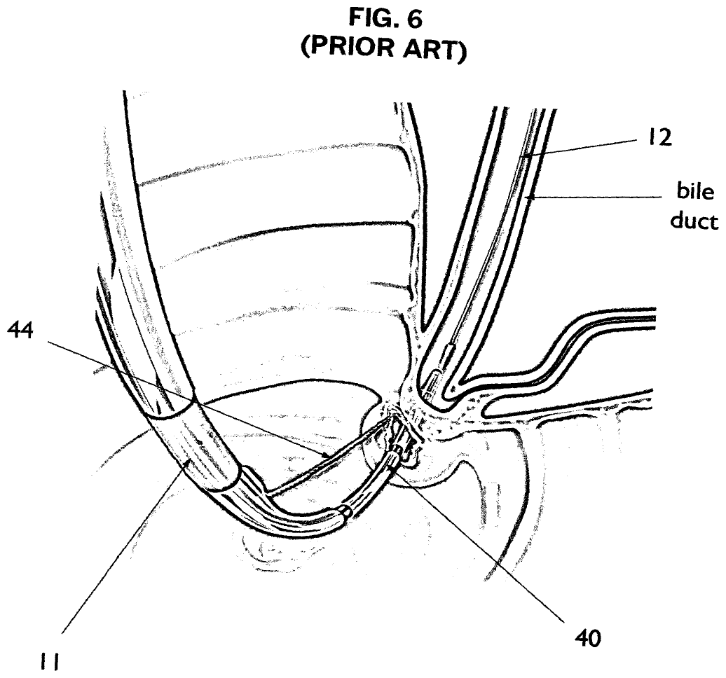

For example, the tool may comprise a tubular lumen and various instruments that may be disposed within the tubular lumen, such as a wire, cannula, catheter or sphincterotome, for example, as illustrated in FIG. 6.

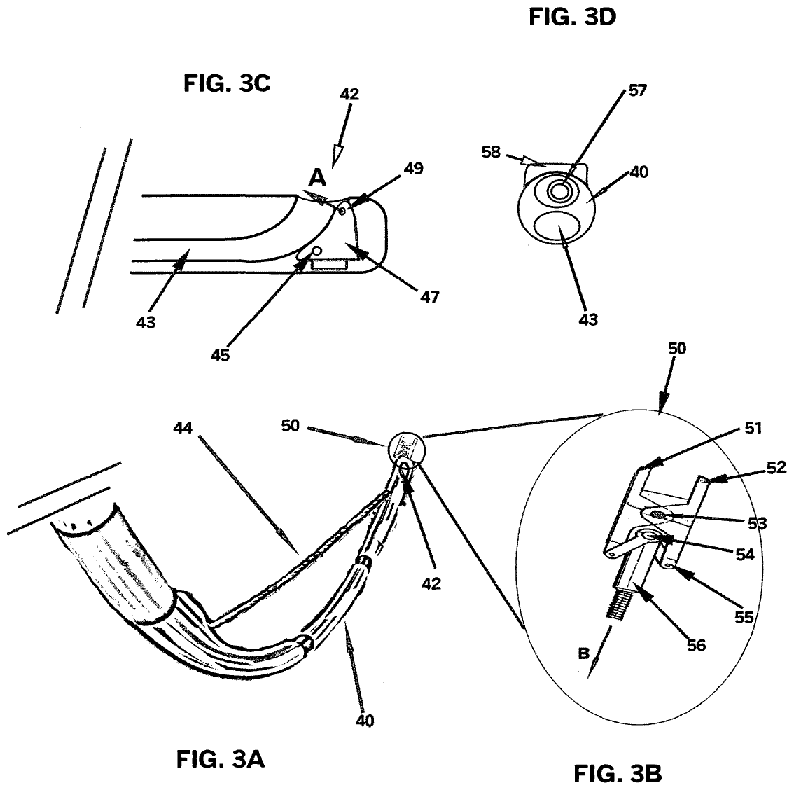

FIG. 3A-3D illustrates an example of a distal end or tip of a tool with (A) a bending portion of the tubular lumen with a grasping device 50 extending from the tip, (B) a detailed view of a mechanism for closing a pair of jaws of a grasping device, (C) a partial cross sectional view along an axis of the lumen showing an orifice on the side of tubular lumen/tip, and (D) an example showing a coupling member 57 and a mounting bracket 58 for the grasping jaws and an orifice extending through the tip without a bend for directing an instrument transversely. FIG. 3C illustrates an example of a tip with an orifice 42 on the side of the tubular lumen/tip. In this cross sectional view along the longitudinal axis, a grasping device is not shown. However, a lumen 43 is shown that could provide room for a second lumen for a grasping device, if desired. In this example, a bending device 47 is shown, which can help to redirect an instrument in any direction with respect to the longitudinal axis of the lumen/tip. For example, as shown, the angle with respect the longitudinal axis is acute, but by rotating the bending device 47 in the reverse direction A, the angle may be increased to perpendicular or even obtuse. For example, a filament may be attached at a coupling point 49 to pull the bending device 47 in reverse direction A by pivoting the entire bending device 47 about a pivot point 45.

FIG. 3A illustrates an example of a portion of a sphincterotome having a bending portion 40 further comprising an orifice 42 on the side of the tubular lumen I tip and a grasping device 50 disposed on the end of the tip and coupled to a manipulator by a filament 44. FIG. 3B is a detailed view of an example of a grasping device 50. The grasping device 50 has a threaded coupler 56, which is capable of coupling to a similarly-threaded female couple of a filament. The manipulator may be coupled to the filament such that when the filament is pulled in direction B, the jaws 51, 52 close, and when pushed in the opposite direction, the jaws 51, 52 open. In this example, the coupler 56 is pivotably coupled at pivot points 54 to a pair of rocker arms, each of which ar□ pivotably attached to a coupling member extending from the jaws at pivot points 55. The coupling members are integrally formed with the pair of jaws 51, 52, and the jaws 51, 52 are pivotably coupled at pivot point 53, which also connects the grasping device 50 to a support 58, which couples the grasping device 50 to the tubular lumen/tip of the tool.

FIGS. 3A-3D illustrates schematically a tool that allows for improved guidance of a cannula in the area of the ampulla of Yater. FIG. 3D shows an alternative utilizing a tip without an orifice on the side. Instead, the orifice is disposed on the end of the tip. So, an instrument passing through the orifice would not be redirected transversely to the lumen. An instrument may assist in teasing the sphincter of oddi open and better align the distal end for entry of wire, cannula or catheter into the bile duct, without damaging the pancreatic duct. A distal portion of the instrument may have a bending section 40 including grasping jaws 50 coupled to one or more controlling members 44 for opening and closing one or both of the members 51, 52 comprising the grasping jaws 50. The bending section 44 may include a filament 44 exiting the bending section 44 at an orifice and proceeding outside of the bending section to a coupling point adjacent or at the end of the bending section, proximate the grasping jaws 15. The angle of connection of the grasping jaws to the bending section 44 may be any angle, and an orifice, proximate the grasping jaws 50 may be provided for a wire, cannula, snare or catheter to extend from the orifice, whether the orifice 42 extends from the side, as shown in FIGS. 3A and 3C or passes through the tip, as shown in FIG. 3D.

The filament 44 may be coupled to a manipulator at a proximal end of the tool for a surgeon to activate the manipulator to pull the filament, bending the bending section 40 at any desired angle relative to the longitudinal axis of the endoscope. Tension pulling on the filament 44 may be controlled to adjust flexure of the bending section 40, as shown for the sphincterotome in FIG. 3A, for example.

Filament 44 may have a portion extending externally to a tubular member of the bending section. Internal to the flexible end is an instrument 12, such as a wire, cannula, snare loop or catheter. FIG. 3A illustrates a flexible end wherein the instrument 12 exits the distal orifice 42 of the flexible end. The orifice may be disposed as shown in FIG. 3C such that the instrument extends transversely to the longitudinal axis of the tip.

In one example, an instrument 12 is directed through the papilla of the sphincter of oddi and into the common bile duct as shown in FIG. 6 (for the prior art). Alternatively, the instrument may be disposed to enter the pancreatic duct, for example, by reorienting the tip. For example, the distal end or tip of the cannulation tool may be disposed within a patient's ampulla of Vater. The open grasping jaws may be positioned at a rim of the papilla, preferably the upper rim proximate the lumen, and the grasping jaws may be closed on the rim of the papilla, grasping the papilla. With the grasping jaws engaged to stabilize the instrument, traction may be applied by movement of the instrument and the endoscope better positioning the orifice of the tip of the bending section and teasing open the papilla to align its lumen for insertion of the instrument.

In one example of the method, the method comprises the following steps: placing the distal tip of the instrument within a patient's ampulla of Vater; disposing the grasping jaws in an open position at a location such that closing the grasping jaws will grasp a portion of the papilla proximate the lumen leading to the common bile duct; closing the grasping jaws such that a portion of the upper rim of the papilla is grasped with the grasping jaws; applying traction to the portion of the papilla with the grasping jaws to position the orifice of the soft tip of the instrument adjacent to the papilla and teasing open the papilla and aligning the lumen of the papilla for insertion of the instrument into the lumen of the papilla; and advancing the instrument into the lumen of the papilla.

For example, the method may advance a cannula or catheter into the lumen of the papilla. In one example, the instrument comprises a bending section, and the bending section is arcuately bent by pulling on a filament coupled on a distal end adjacent to the tip of the instrument and on its opposite proximal end to a manipulator capable of applying and releasing tension on the filament. For example, a longitudinal axis of the tip may be directed at an angle such that the instrument extending along the longitudinal direction extends through the lumen of the papilla and into the common bile duct without damaging the pancreatic duct. Alternatively, an orifice may be disposed on a side of the tip such that the instrument extends transversely from the longitudinal axis of the tip and through the lumen of the papilla. In one example, the orientation of the orifice on the side of the tip may be arranged to either extend the instrument into the common bile duct or the pancreatic duct, without damaging the pancreatic duct.

In one example, the portion of the papilla grasped by the grasping jaws is the upper rim of the papilla. In one example, a method comprises: placing the distal tip of the instrument within a patient's ampulla of Vater; disposing the grasping jaws in an open position at a location such that closing the grasping jaws will grasp a portion of the papilla proximate the lumen leading to the common bile duct; closing the grasping jaws such that a portion of the upper rim of the papilla is grasped with the grasping jaws; applying traction to the portion of the papilla with the grasping jaws to position the orifice of the soft tip of the instrument adjacent to the papilla and teasing open the papilla and aligning the lumen of the papilla for insertion of the instrument into the lumen of the papilla; applying tension to the filament of the bending portion of the instrument, arcuately bending the bending portion such that the longitudinal axis of the tip of the instrument is favorably angled for penetration into the lumen of the papilla; advancing the tip of the instrument into the lumen of the papilla; and advancing a wire, a cannula, a catheter or a loop along the longitudinal axis of the tip of the instrument through the papilla and into the common bile duct. For example, a cannula is inserted into the common bile duct.

In one example, during a cannulation procedure, involuntary movements occur within the intestinal tract, and the grasping jaws assist in at least partially anchoring the tip of the instrument such that the tip of the instrument remains in position relative to the lumen through the papilla.

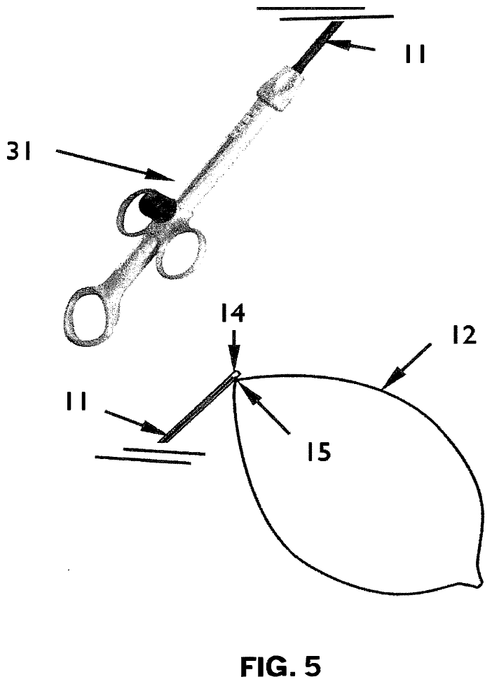

FIG. 5 illustrates an example of a soft tip 14 having an orifice 15 disposed transversely to the longitudinal axis of the tip. For example, the tip 14 comprises a soft blunt end, which may be made of any resilient or elastomeric material. In preferred material is a silicone rubber or a silicone rubber coating on a blunt substrate. A side orifice 15 for directing an instrument from the tubular lumen of the instrument is shown with a snare loop 12 extending transversely. As the instrument 12, such as a snare loop, extends outwardly from the orifice 15 of the tubular lumen it extends in a transverse direction to the longitudinal axis of the tubular lumen. Another example, includes a method utilizing an instrument including a blunt tip and side opening that includes a filament in communication with a manipulator 31 to further assist in performing endoscopic intervention.

FIG. 5 illustrates a distal end with the wire loop in an open position. The tubular lumen 11 includes a snare loop coupled to a manipulator by a control member, such as a wire or cable for opening and closing the wire loop. The snare loop is coupled to the cable by a flexible joint, in this example, which is capable of angularly extending the wire loop from the orifice of the tubular lumen. The tubular lumen 11, in this example, has a distal, soft blunt tip 14 and an orifice 15 disposed adjacent to the tip 14. For example, the orifice 15 may be disposed 1-3 mm from the end of the tip 14. In one example, the orifice 15 is disposed 2 mm from the end of the tip 14.

A primary control filament, such as a cable, is shown in FIG. 3A and FIG. 5. Its proximal end is coupled to a manipulator 31 for pushing and pulling the control filament, which in the example of FIG. 5 opens and closes a snare loop extending transversely from the orifice 15 disposed on the side of the lumen/tip 14. Outward and inward movement of the snare loop 12 opens and closes the snare loop 12, respectively, allowing the snare loop 12 to be used for dissection of a bolus, for example.

For example, the distal end of the tip 14 prevents damage to tissues of the patient. Since the snare loop 12 does not extend out of the tip 14, the tip may be made of any soft materials such as an elastomeric material. In one example, the tip is of a soft silicone rubber, which prevents damage to soft tissues, even when the tip is disposed between a bolus and a mucus membrane. This makes positioning of the snare loop 12 much easier and reduces unintended complications.

The tip of the tool is soft and blunt and capable of resting on the gastrointestinal wall to stabilize the instrument, such as during removal of a bolus impaction within the esophagus. In one example, a method comprises: endoscopically inserting an instrument having a soft blunt tip into an esophagus of a patient; disposing the soft blunt tip adjacent a bolus impaction; manipulating a manipulator such that a snare loop extends transversely relative to a longitudinal axis of the soft blunt tip, the snare loop extending from an orifice disposed on a side of a tubular lumen or the soft blunt tip or a combination of these; and using the snare loop to dislodge the bolus by dissection or removal of the bolus or both dissection and removal of the bolus.

This detailed description provides examples including features and elements of the claims for the purpose of enabling a person having ordinary skill in the art to make and use the inventions recited in the claims. However, these examples are not intended to limit the scope of the claims, directly. Instead, the examples provide features and elements of the claims that, having been disclosed in these descriptions, claims and drawings, may be altered and combined in ways that are known in the art.

What is claimed is:

1. An endoscopic tool for use with an endoscope for surgical procedures within a body cavity comprises:
a manipulator disposed at a proximal end of the tool for controlling the tool;
a primary filament coupled to the manipulator by a proximal end of the primary filament;
an instrument disposed at a distal end of the tool and coupled with the primary filament, the primary filament having a stiffness sufficient to both push and pull the instrument;
an elongated tubular lumen has a longitudinal axis, and at least a portion of the primary filament extends through the tubular lumen along the longitudinal axis of the tubular lumen, and the instrument is disposed within the tubular lumen in a fully retracted position and extends from the tubular lumen in a fully extended position;
a blunt tip comprises a soft material, whereby biological tissues within the body cavity are not damaged when the tip is pressed against the tissues, and the blunt tip is coupled to the tubular lumen;
an orifice is disposed on a side wall of the tubular lumen or the blunt tip and is arranged such that the instrument extends from the orifice in the fully extended position transversely with respect to the longitudinal axis of the tubular lumen.

2. The tool of claim 1, wherein the filament is a cable or wire.

3. The tool of claim 1, wherein the blunt tip is of a silicone rubber material.

4. The tool of claim 1, wherein the instrument is a snare loop.

5. The tool of claim 1, further comprising a gripping device having jaws, wherein the gripping device is coupled to the tip and arranged on the tip such that the jaws of the gripping device extends beyond the tip, wherein the gripping device is operatively coupled to the manipulator such that the manipulator may open and close the jaws of the gripping device.

6. The tool of claim 5, wherein the jaws have a surface and the surface is coated with an elastomeric material.

7. The tool of claim 6, wherein the elastomeric material is a silicone.

8. The tool of claim 5, wherein the jaws of the gripping device are arranged such that the jaws close when a scissor mechanism of the gripping device is activated by pulling on a filament coupled to the scissor mechanism.

9. The tool of claim 8, wherein the scissor mechanism comprises pair of jaws joined together at a pivot point by a fastener and are coupled to a member fixedly attached to the tip of the tool.

10. The tool of claim 8, wherein each of the pair of jaws have a coupling member extending rearwardly from a mouth formed by the jaws, and each coupling member is attached at a pivot point to a first end of each of a pair of rocker arms, and the opposite end of each of the pair of rocker arms is coupled pivotally to a coupling element, and the coupling element is coupled to a distal end of a connecting filament, and the proximal end of the connecting filament is coupled the manipulator, such that the filament may be pushed and pulled by the manipulator, opening and closing the pair of jaws of the grasping device.

11. The tool of claim 5, wherein the instrument is a wire, a cannula or a catheter.

12. The tool of claim 11, wherein the instrument is a catheter.

13. The tool of claim 5, wherein the tubular lumen comprises a bending portion, wherein the bending portion is coupled to the manipulator by a secondary filament, and applying tension to the secondary filament bends the end of the tubular lumen when the tubular lumen is extended from the endoscope.

14. The tool of claim 13, wherein the tip is oriented at an angle greater than 90 degrees relative to the longitudinal axis of a remaining portion of the tubular lumen that remains unbent.

15. A method of using the device of claim 14 comprising:
placing the blunt tip of the tool within a patient's ampulla of Vater;
disposing the jaws of the grasping device in an open position at a location such that closing the jaws will grasp a portion of the papilla proximate the lumen leading to the common bile duct;
closing the jaws such that a portion of the upper rim of the papilla is grasped with the grasping jaws;
applying traction to the portion of the papilla with the grasping jaws to position the orifice of the soft tip of the instrument adjacent to the papilla and teasing open the papilla and aligning the lumen of the papilla for insertion of the instrument into the lumen of the papilla; and
advancing the instrument into the lumen of the papilla.

16. The method of claim 15, wherein a cannula or catheter is advanced into the lumen of the papilla.

17. The method of claim 16, wherein the bending portion is arcuately bent by pulling on the secondary filament using the manipulator.

18. The method of claim 17, wherein the longitudinal axis of the tip is redirected by the bending portion at an angle such that the instrument extending along the longitudinal direction extends through the lumen of the papilla and into the common bile duct without damaging the pancreatic duct.

19. The method of claim 17, wherein the orifice on the side of the tubular lumen, the tip or both thereof is disposed such that the instrument extends transversely from the longitudinal axis of the tip and through the lumen of the papilla.
